(19) United States
(12) Patent Application Publication
(10) Pub. No.: US 2004/0115270 A1
(43) Pub. Date: Jun. 17, 2004

(54) Absorption and controlled release of polyethers from hydrogel biomaterials

(76) Inventors: Dharmendra Jani, Fairport, NY (US); Joseph C. Salamone, Fairport, NY (US); Zhenze Hu, Pittsford, NY (US); Erning Xia, Penfield, NY (US); Roya Borazjani, Rochester, NY (US); Daniel M. Ammon JR., Rochester, NY (US)

Correspondence Address: BAUSCH & LOMB INCORPORATED, ONE BAUSCH & LOMB PLACE, ROCHESTER, NY, 14604-2701, US

(21) Appl. No.: 10/319,132

(22) Filed: Dec. 13, 2002

(51) Int. Cl. A61K 9/14

(52) U.S. Cl. 424/486

(57) ABSTRACT

The present invention is directed to an ophthalmic solution for soft contact lenses for controlled release of polyethers into an eye's tear film. Polyether components of the subject ophthalmic solution are released from the soft contact lens material matrix over long time periods to produce longer lasting wetting performance, improved lubricity, improved end-of-the-day comfort and reduced feeling of dryness from wearing contact lenses.

### FIELD OF THE INVENTION

The present invention relates to an ophthalmic solution and method for absorption and controlled release of components of the solution by hydrogel biomaterials. More particularly, the present invention relates to an ophthalmic solution comprising polyethers that exhibit ready absorption into hydrogel biomaterials, such as that of a contact lens, and slow release over a period of time in an aqueous environment for longer lasting wetting performance.

### BACKGROUND OF THE INVENTION

Contact lenses in wide use today fall into two categories. First, there are the hard or rigid corneal type lenses that are formed from materials prepared by the polymerization of acrylic esters, such as poly(methyl methacrylate) (PMMA). Secondly, there are the gel, hydrogel or soft type of lenses made by polymerizing such monomers as 2-hydroxyethyl methacrylate (HEMA) or, in the case of extended wear lenses, made by polymerizing silicon-containing monomers or macromonomers. Solutions that wet the lenses before insertion into the eye are required for both the hard and soft types of contact lenses,

although the formulations of the solutions have tended to differ based on the different desired properties of the solutions. After the contact lenses are inserted in the eye, ophthalmic solutions for rewetting, lubricating, and/or enhancing wearer comfort are sometimes applied to the eye by means of a drop dispenser.

Isotonic solutions for improving the comfort of wearing soft contact lenses by being added directly to the contact lens while in the eye are known. Such solutions typically contain viscosity enhancing agents, lubricants, surfactants, buffers, preservative, and salts. For example, Sherman discloses in U.S. Pat. No. 4,529,535 a rewetting solution that is particularly useful for rigid silicone copolymer contact lenses, including extended wear lenses. In one embodiment, the rewetting solution contains the combination of hydroxyethylcellulose, poly(vinyl alcohol) and poly(N-vinylpyrrolidone).

Ogunbiyi et al. disclose in U.S. Pat. No. 4,786,436 a wetting solution comprising collagen and other demulcents such as hydroxyethylcellulose, methylcellulose, carboxymethylcellulose, hydroxypropylmethylcellulose, hydroxylpropylcellulose and the like.

Su et al. disclose in U.S. Pat. No. 4,748,189 ophthalmic solutions for improving the exchange of fluid in the area outside a hydrogel contact lens in the area underneath the hydrogel contact lens in order to permit tear exchange to occur, thereby preventing the accumulation of waste matter and debris under the lens. The solution contains a hydrogel flattening agent, for example, urea, glycerin, propylene glycol, sorbitol, or an amino-ethanol. Surfactants that are useful in the solution include poloxamer and tyloxapol. Suitable lubricants include hydroxyethylcellulose, poly(vinyl alcohol) and poly(N-vinylpyrrolidone).

Winterton et al. disclose in U.S. Pat. No. 5,209,865 a conditioning solution for contact lenses that comprises a combination of a poloxamine and a poloxamer surfactant, each having an HLB (hydrophilic-lipophilic balance) of seven or below. The solution according to the invention forms a uniform hydrophilic film on a lens surface for which proteins have very little affinity. As such, a contact lens contacted by the solution is said to have a coating that provides a prophylactic effect to the lens.

Zhang et al. disclose in U.S. Pat. No. 5,604,189 and U.S. Pat. No. 5,773,396 a composition for cleaning and wetting contact lenses comprising (i) a non-amine polyethyleneoxy-containing compound having an HLB of at least about 18, (ii) a surface active agent having cleaning activity for contact lens deposits that may have an HLB less than 18, and (iii) a wetting agent. Such compositions can include, as the wetting agent, an ethoxylated glucose derivative such as glucam as also disclosed in U.S. Pat. No. 5,401,327 to Ellis et al. Tyloxapol is a conventional surface active agent, used for example in Allergan's Complete™ multipurpose solution, which agent has cleaning activity for contact-lens deposits and has an HLB less than 18.

Unlike hard lenses, the soft type of contact lenses have a tendency to bind and concentrate significantly more fluids, environmental pollutants and water impurities. Likewise, the soft type of contact lenses is more susceptible to the deposition of protein or lipids or both. Thus, the use of enzymes or equivalent protein-removing agents has been conventionally employed for weekly or daily protein removal from worn lenses. In contrast, surfactant cleaning agents in daily lens care solutions are useful for the removal of lipid or lipid-like materials from the lenses. With the advent of extended wear lenses, however, in which lenses are worn overnight and even continuously over a plurality of whole days, night and day, the lens wearers no longer have the opportunity to remove, by means of the conventional lens care solutions, the depositions that have accumulated over the, day.

It would, therefore, be desirable to have an ophthalmic solution that could be applied to a contact lens that not only rewets the lens but also provides controlled release wetting of the lens over a period of time until such lens is removed from the eye and cleaned or disposed.

### SUMMARY OF THE INVENTION

The present invention relates to an ophthalmic solution and method for absorption and controlled release of components of the solution by hydrogel biomaterials such as for example hydrogel biomaterials in the form of soft contact lenses. The ophthalmic solution of the present invention comprises polyethers based upon poly(ethylene oxide)-poly(propylene oxide)-poly(ethylene oxide), i.e., (PEO-PPO-PEO), or poly(propylene oxide)-poly(ethylene oxide)-poly(propylene oxide), i.e., (PPO-PEO-PPO). PEO-PPO-PEO and PPO-PEO-PPO are commercially available under the trade names Pluronics™, R-Pluronics™, Tetronics™ and R-Tetronics™ (BASF Wyandotte Corp., Wyandotte, Mich.). Polyethers of the subject ophthalmic solution exhibit ready absorption into hydrogel biomaterials such as those used in the manufacture of soft type contact lenses. Polyethers of the subject ophthalmic solution after absorption to a high concentration exhibit slow release from the hydrogel biomaterials over a period of time in an aqueous environment. In accordance with the present invention, the polyethers release slowly from a worn contact lens into an eye's tear film over a long time period to produce longer lasting wetting performance, improved lubricity, improved end-of-the-day comfort and reduced feeling of dryness from wearing contact lenses. The subject ophthalmic solutions are likewise suitable for use as lens packaging solutions.

Accordingly, it is an object of the present invention to provide an ophthalmic solution that provides longer lasting wetting performance for contact lenses.

Another object of the present invention is to provide a method for using an ophthalmic solution to provide longer lasting wetting performance for contact lenses.

Another object of the present invention is to provide an ophthalmic solution and a method for using the same that improves contact lens lubricity and end-of-the-day comfort.

Another object of the present invention is to provide an ophthalmic solution and method for using the same that reduces the feeling of eye dryness from wearing contact lenses.

Another object of the present invention is to provide an ophthalmic solution with components that exhibit ready absorption into hydrogel biomaterials.

Still another object of the present invention is to provide an ophthalmic solution with components that release slowly from hydrogel biomaterials into an aqueous environment.

These and other objectives and advantages of the present invention, some of which are specifically described and others that are not, will become apparent from the detailed description and claims that follow.

### BRIEF DESCRIPTION OF THE DRAWINGS

FIG. 1 is a graph of Group I dynamic contact angle hysteresis;

FIG. 2 is a graph of Group I surface tension of probe medium;

FIG. 3 is a graph of Group IV dynamic contact angle hysteresis;

FIG. 4 is a graph of Group IV surface tension of probe PBS;

FIG. 5 is a graph of Group I controlled release of 1 percent solutions;

FIG. 6 is a graph of Group I controlled release of 5 percent solutions;

FIG. 7 is a graph of Group IV controlled release of 1 percent solutions;

FIG. 8 is a graph of Group IV controlled release of 5 percent solutions;

FIG. 9 is a graph of Group I controlled release of wetting agents;

FIG. 10 is a graph of Group III controlled release of wetting agents;

FIG. 11 is a graph of Group IV controlled release of wetting agents;

FIG. 12 is a graph of Group I coefficient of friction in various solutions;

FIG. 13 is a graph of Group III coefficient of friction in various solutions;

FIG. 14 is a graph of Group IV coefficient of friction in various solutions; and

FIG. 15 is a graph of polyether absorption in Group IV lenses.

### DETAILED DESCRIPTION OF THE INVENTION

The present invention relates to an ophthalmic solution and method of use for absorption and controlled release of components of the solution by hydrogel biomaterials such as for example hydrogel biomaterials in the form of soft contact lenses. The ophthalmic solution of the present invention preferably comprises greater than approximately 1 percent by weight polyethers based upon poly(ethylene oxide)-poly(propylene oxide)-poly(ethylene oxide), i.e., (PEO-PPO-PEO), or poly(propylene oxide)-poly(ethylene oxide)-poly(propylene oxide), i.e., (PPO-PEO-PPO). PEO-PPO-PEO and PPO-PEO-PPO are commercially available under the trade names Pluronics™, R-Pluronics™, Tetronics™ and R-Tetronics™ (BASF Wyandotte Corp., Wyandotte, Mich.). More preferably, the ophthalmic solution of the present invention comprises approximately 1.5 to 14 weight percent and most preferably between approximately 2 to 5 weight percent polyethers. Polyethers of the subject ophthalmic solution exhibit ready absorption into hydrogel biomaterials such as those used in the manufacture of soft type contact lenses. The subject absorption of polyethers into the material matrix of a contact lens described herein differs from the adsorption of surfactants onto the surface of a contact lens as disclosed by Salpekar et al., U.S. Pat. No. 6,440,366. The visual quality and acquity of the hydrogel biomaterials is not affected by the absorption of the solution polyethers. Polyethers of the subject ophthalmic solution, after absorption to a high concentration by a hydrogel biomaterial, exhibit slow release from the hydrogel biomaterial over a period of time in an aqueous environment. In accordance with the present invention, the polyethers release slowly from a worn contact lens into an eye's tear film over a long time period to produce longer lasting wetting performance, improved lubricity, improved end-of-the-day comfort and reduced feeling of dryness from wearing contact lenses.

In accordance with the present invention, a sterile ophthalmically safe aqueous storage solution is used for treating contact lenses prior to placement in the eye or by administering in the form of drops in the eye, or is used for packaging contact lenses. Solutions of the present invention have a pH of about 6.0 to 8.0, preferably about 6.5 to 7.8. Suitable buffers may be added to the subject solutions such as but not limited to boric acid, sodium borate, potassium citrate, citric acid, sodium bicarbonate, and various mixed buffers. Generally, buffers will be used in amounts ranging from about 0.05 to 2.5 percent by weight, and preferably from 0.1 to 1.5 percent by weight.

Typically, the ophthalmic solutions of the present invention include at least one tonicity adjusting agent, optionally in the form of a buffering agent, for providing an isotonic or close to isotonic solution such that the osmolality is about 200 to 400 mOsm/kg, preferably about 250 to 350 mOsm/kg. Examples of suitable tonicity adjusting agents include but are not limited to sodium and potassium chloride, dextrose, glycerin, calcium and magnesium chloride. These agents are typically used individually in amounts ranging from about 0.01 to 2.5 weight percent and preferably from about 0.2 to about 1.5 weight percent.

It may also be desirable to optionally include in the subject solutions water soluble viscosity builders such as for example but not limited to poly(vinyl alcohol). Because of their demulcent effect, viscosity builders have a tendency to further enhance the lens wearer's comfort by means of a film on the lens surface cushioning impact against the eye.

The subject solutions are sterilized by heat and hermetically sealed. If used as a contact lens packaging solutions, the solution is sterilized by heat and hermetically sealed in a blister pack with a contact lens. The subject solutions, if heat sterilized and hermetically sealed, may be used in the absence of a germicide compound.

Dynamic contact angle analysis was used to determine the extent of wettability produced by different ophthalmic lens care multipurpose solutions. Two contact lens materials were used in the ophthalmic solution wettability study as set forth in Table 1 below.

TABLE 1
Contact Lens Materials

| Sample | Components | Weight Percent |
|---|---|---|
| Group I | HEMA (2-hydroxyethyl methacrylate) | 84.10 |
| | Glycerin | 14.92 |
| | EGDMA (ethylene glycol dimethacrylate) | 0.98 |
| Group IV | HEMA | 84.08 |
| | EGDMA | 0.11 |
| | Methacrylic acid | 2.61 |
| | BME (benzoin methyl ether) | 0.17 |
| | Dimethylformamide | 13.03 |

The different ophthalmic lens care multipurpose solutions used to determine the extent of wettability in the dynamic contact angle analysis are set forth below in Table 2.

TABLE 2
Ophthalmic Lens Care Multipurpose Solutions

| Test Solution | Product | Components | Weight Percent |
|---|---|---|---|
| C | Renu MultiPlus | Tetronic 1107 | 1.0 |

| Test | | |
|---|---|---|
| Solution | Components | Weigh Percent |
| A | Boric acid | 0.85 |
| | Sodium Phosphate (Monobasic) | 0.15 |
| | Sodium Phosphate (Dibasic) | 0.31 |
| | Sodium Chloride | 0.26 |
| | HAP (30%) (hydroxyalkyl phosphonate) | 0.10 |
| | Tetronic 1107 | 1.00 |
| | Pluronic F127 | 2.00 |
| | Polymer JR | 0.02 |
| | PHMB(20%) (polyhexamethylene biguanide) | 1.1 ppm |
| B | Boric Acid | 0.85 |
| | Sodium Phosphate (Monobasic) | 0.15 |
| | Sodium Phosphate (Dibasic) | 0.31 |
| | Sodium Chloride | 0.36 |
| | HAP (30%) | 0.10 |
| | Tetronic 1107 | 1.00 |
| | Pluronic F127 | 2.00 |
| | Polymer JR | 0.02 |
| | PHMB(20%) | 1.1 ppm |

The dynamic contact angle analysis used to determine the extent of wettability produced by different ophthalmic lens care multipurpose solutions is described in still greater detail in the example that follows.

### EXAMPLE 1

A. Sample Preparation

Group I: HEMA films were UV cast polymerized around a square glass cover slip to provide a flat substrate for conducting a dynamic contact angle study. The dimensions of the prepared substrates were 22 mm×22 mm×0.25 mm. The substrates were extracted in hot deionized water for two hours.

Group IV: The ionic monomer mix was UV cast polymerized around a rectangular fluorosilicon acrylate wafer to provide a flat substrate for the dynamic contact angle study. The dimensions of the substrate were approximately 12 mm×25 mm×1 mm. The substrates were extracted in phosphate buffered saline* (PBS) overnight at 37° C.

| *Phosphate buffered saline = sodium phosphate (monobasic) | | 0.016% |
|---|---|---|
| | sodium phosphate (dibasic) | 0.066% |
| | sodium chloride | 0.88% |
| | deionized water | 93.038% |

B. Dynamic Contact Angle Study

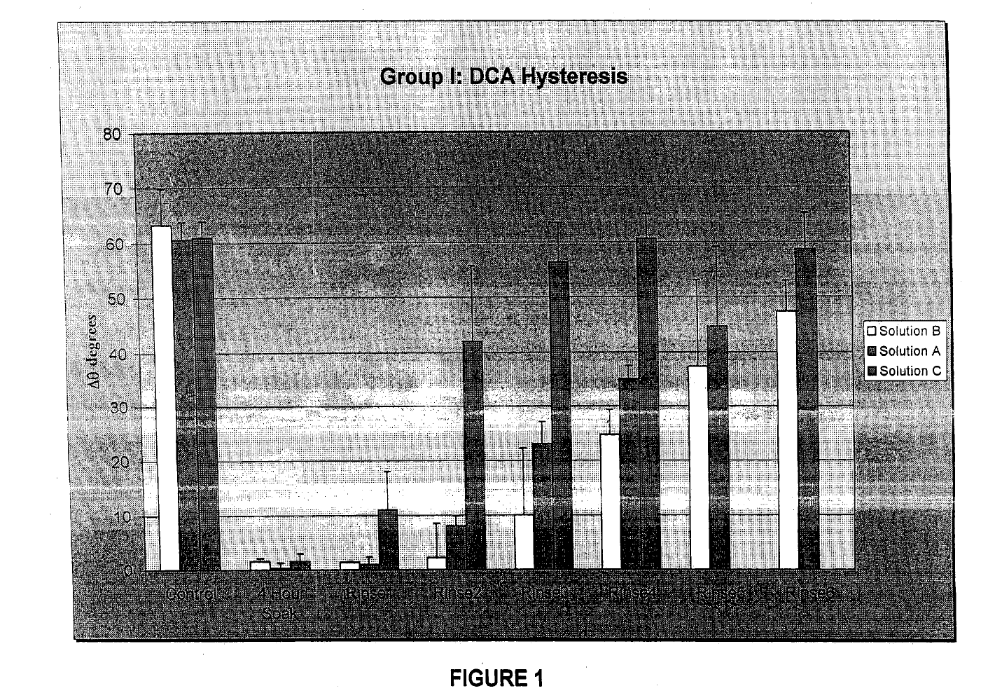

Group I: Each HEMA substrate was suspended inside a CAHN DCA 315 apparatus. Dynamic contact angles and the contact angle hysteresis were measured using the Wilhelmy Plate method by alternatively inserting and withdrawing the flat substrate into and out of PBS at approximately 32° C. which was used as control. For each test, the sample was inserted and withdrawn twice (two cycles) in the probe medium. A sample of the wetting force experienced by the substrate in the probe medium is as shown in FIG. 1. The surface tension of the probe medium was also measured using the DuNouy Tensiometer ring method.

The substrate was soaked for four hours in a test solution and the dynamic contact angles measured as described above. The HEMA substrate was then rinsed by dipping twenty-five times in 80 ml of PBS (pH=7.27) at approximately 32° C. The dynamic contact angles were again measured as described above in PBS at approximately 32° C. This rinse and contact angle test process was repeated until the substrates reverted to near control state of higher hydrophobicity. The surface tension of the probe medium (PBS solution) was measured as described above.

Group IV: Dynamic contact angles were measured as described above for Group I. Each rinse step involved fifty dips in PBS. The surface tension of probe PBS was measured after each rinse cycle.

C. Results

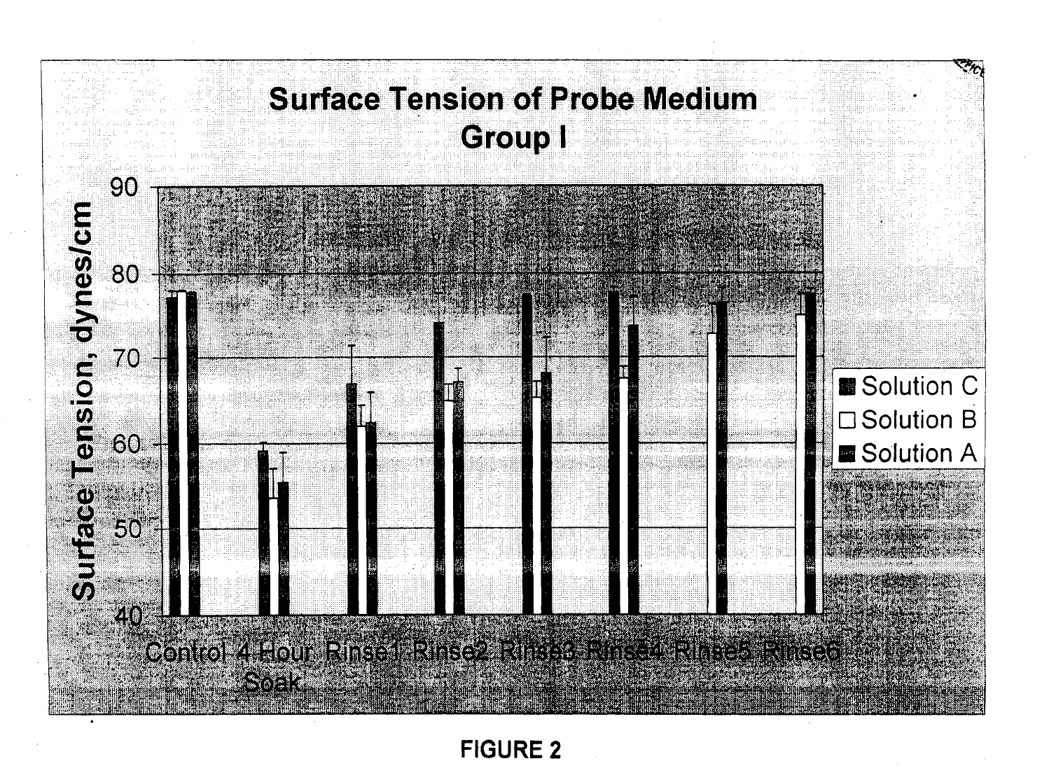

Group I: Results for Group I are illustrated in FIGS. 1 and 2. The smaller the contact angle hysteresis, AO, the better the wettability of the surface. As suggested by the lower contact angle hysteresis after repeated rinses, both test solutions A and B showed better wetting performance than that of test solution C where lower contact angles were obtained even after six rinse cycles (a total of 150 dips). The surface tension values of the probe medium (PBS) support the contact angle results as well. In case of test solution C, the surface tension of probe medium reverted to near PBS (control) value much quicker than for the two test solutions. This suggests that test solutions A and B were absorbed more efficiently into the HEMA matrix and could therefore maintain the wetting ability longer than test solution C through a sustained release of the wetting agents.

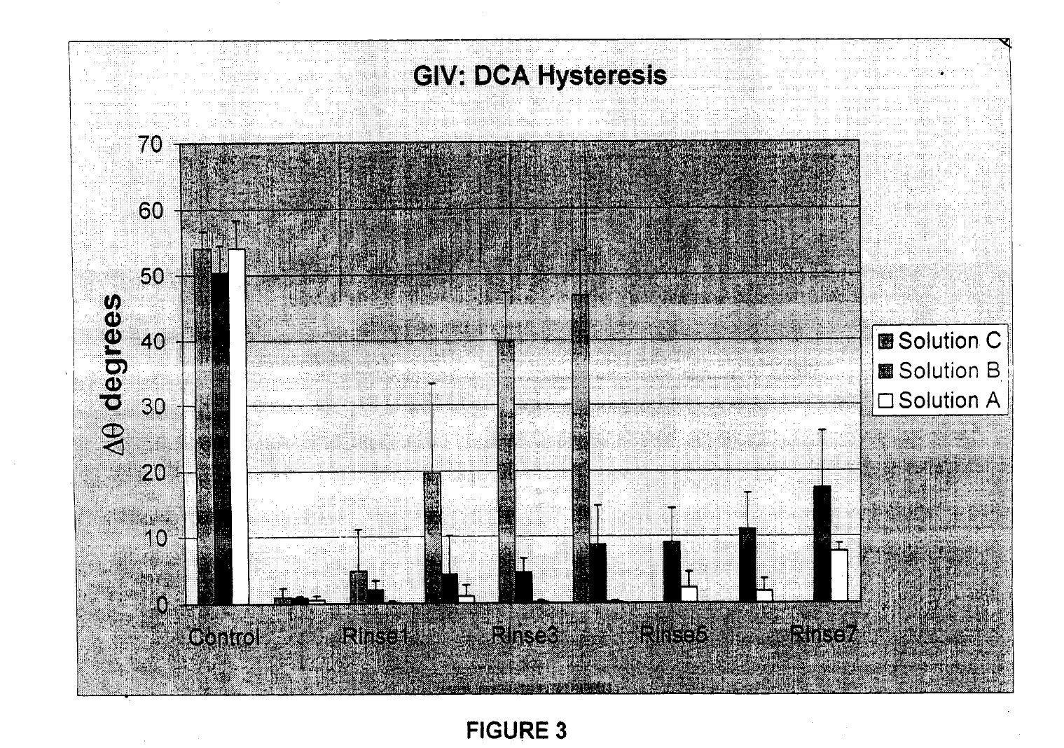

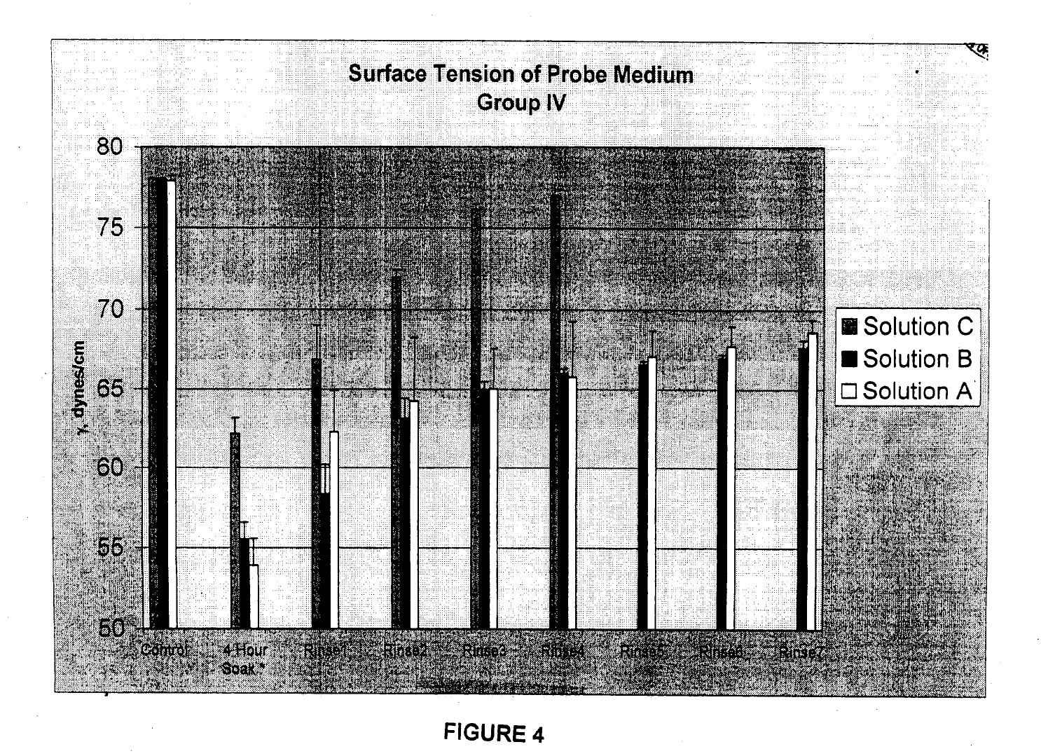

Group IV: Results for Group IV are illustrated in FIGS. 3 and 4. The two test solutions, A and B, performed significantly better than test solution C. The improved and longer lasting wetting performance is most likely attributable to the ionic interactions between pluronic and tetronic and the ionic groups in the substrate. Test solution A exhibited enhanced wetting over solution B, which can be attributed to the lower salt concentration in solution A compared to solution B. This allows the gel matrix to expand more and trap more wetting solution into the matrix. Consequently, the matrix is able to provide a longer sustained release of the wetting agents for increased wettability. The probe medium after each test showed an overall reduced surface tension for solution A and solution B suggesting that the wetting solution is released in greater quantity and over a prolonged period than solution C. All solutions exhibited longer wetting performance for Group IV (ionic) material relative to Group I (non-ionic) material.

D. Conclusion

Based on the Dynamic Contact Angle study, the two test solutions, solution A and solution B, exhibited a longer lasting wetting ability than solution C (1% Tetronic 117) for Group I and Group IV material. There was no significant difference in wetting abilities of the two test solutions for Group 1 material.

An in-vitro study was conducted to determine the rate of release of surfactants from various lens materials after being soaked for four hours in different polyether solutions. The study attempted to simulate the tear turnover rate in the eye by providing a constant supply of buffered saline (PBS) to the lens and collecting the liquid eluting from the lens every hour. Surface tension of the collected volume was measured using a DuNouy ring method. Reduced surface tension relative to the control PBS would indicate the presence of surfactants in the lens. The study of continuous release of polyethers from various lens materials is described in still greater detail in the example that follows.

### EXAMPLE 2

A. Lens Materials

Two materials were used in the subject continuous release of polyethers from various lens materials study as described below.

Group I: Optima™ FW (−3.25 D) (Bausch & Lomb)

Group IV: SureVue™ (−7.00 D) (Johnson & Johnson)

B. Solutions

The solutions used in the subject continuous release of polyethers from various lens materials study are set forth below in Table 3.

TABLE 3

| Solution | Abbreviation |
|---|---|
| Base solution | BS |
| Base solution + Polymer JR | BS + PJR |
| 1% Tetronic 1107 | 1% T |
| 1% Pluronic F127 | 1% P |
| 1% Tetronic/Pluronic | 1% T/P |
| 5% Tetronic 1107 | 5% T |
| 5% Pluronic F127 | 5% P |
| 5% Tetronic/Pluronic | 5% T/P |

C. Procedure

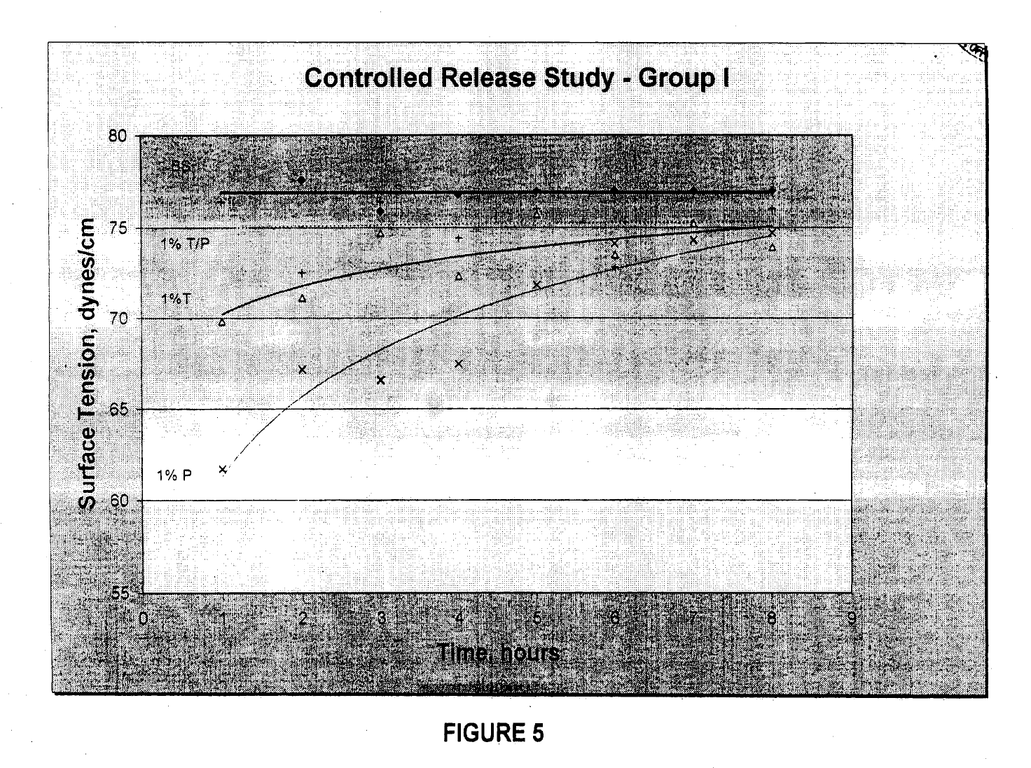

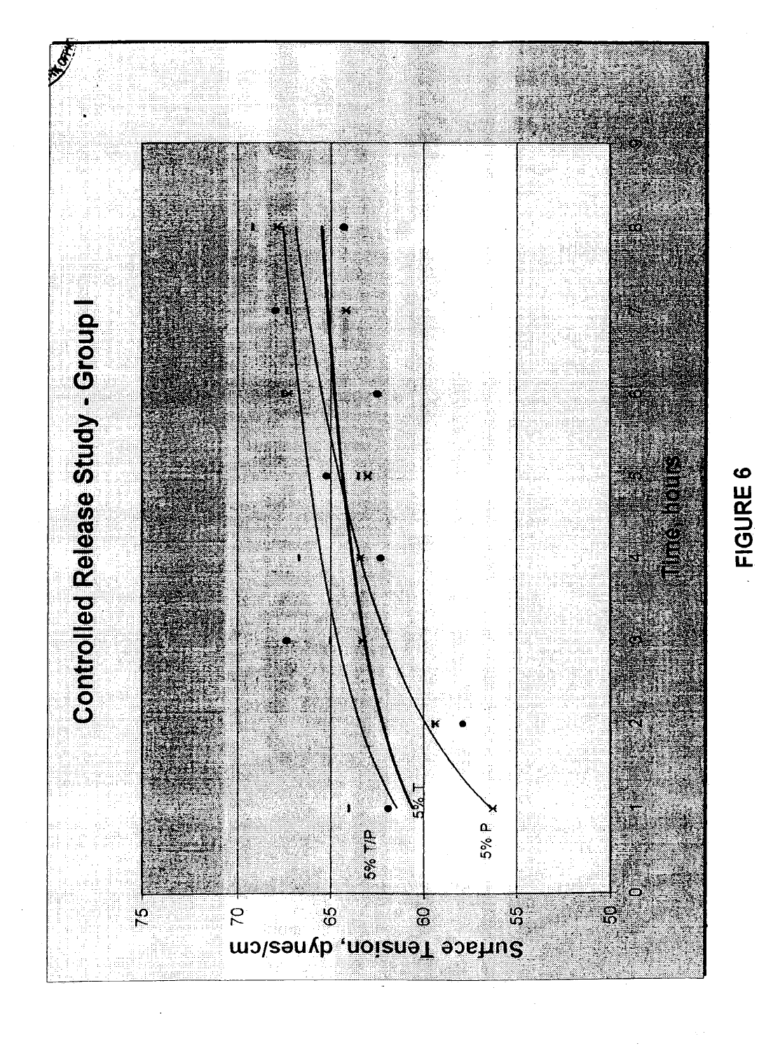

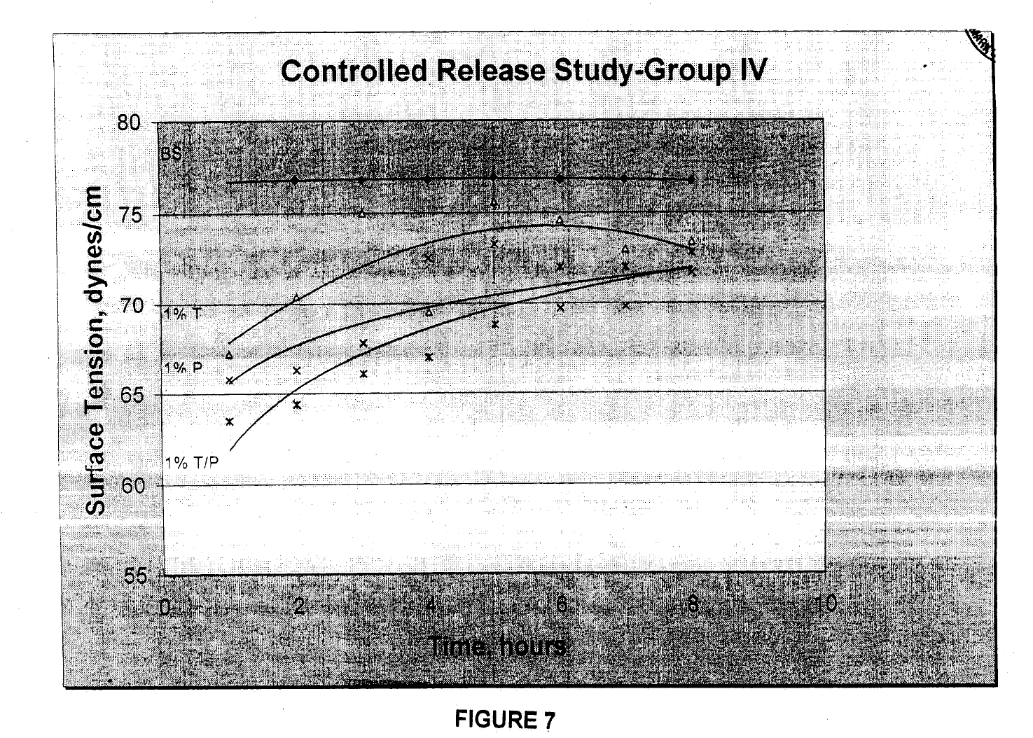

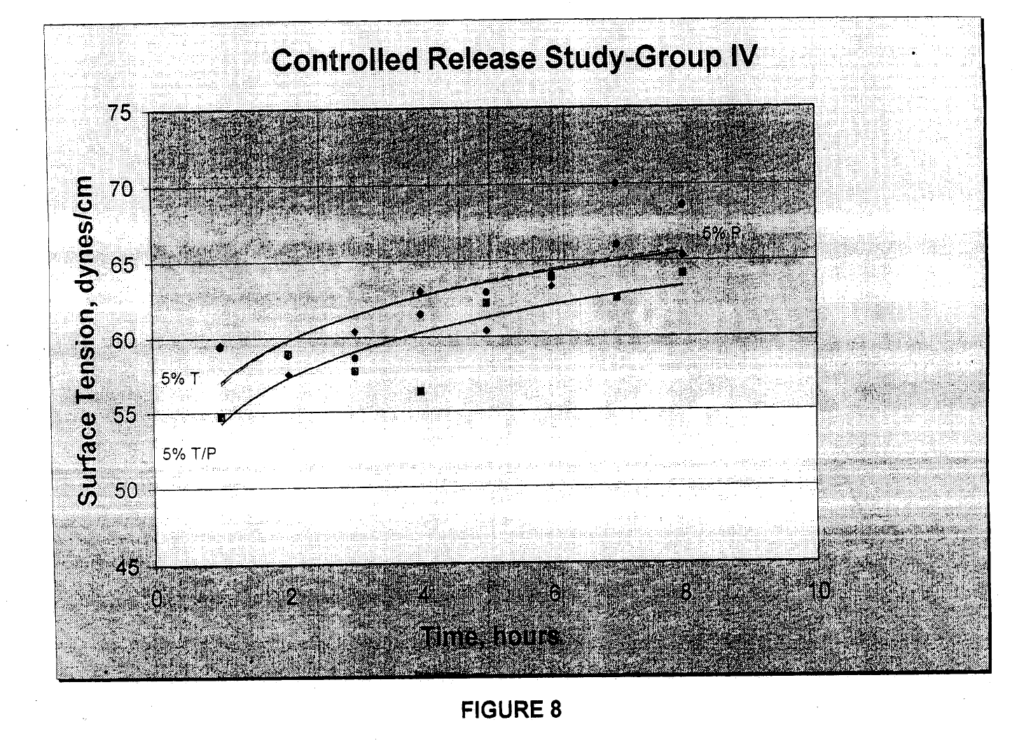

Lenses of Group I and Group IV type were soaked for four hours in the various polyether solutions. The lenses were then removed and placed in a lens basket designed to receive a continuous infusion of phosphate buffered saline (PBS). A micro-infusion pump delivered 3.8 μl/min of PBS continuously to the lens surface for 18 hours to simulate the human tear film secretory rate in the eye. The solution dripping off the lens was collected over every hour for eight hours in a closed container to prevent evaporation. This volume was diluted with PBS to obtain 25 ml of solution. The apparent surface tension of the resulting solution was measured using the DuNouy ring method and the results were plotted as shown in FIGS. 5 through 8.

D. Results and Conclusions

Non-linear regression models were used to fit the curves to the data collected. Since the surface tension is directly proportional to the concentration of surface active agents and since the elute volume was not exactly the same for each sample collected, some scatter in the surface tension data was expected. However, the trends illustrated in the graphs of FIGS. 5 through 8 are unmistakable.

An in-vitro study was conducted to compare the rate of release of wetting agents from various lens materials after being soaked in various solutions. This study attempted to simulate the tear turnover rate in the eye by providing a constant supply of buffered saline (PBS) to the lens and collecting the liquid eluting from the lens every hour. Surface tension of the collected volume was measured using a DuNouy ring method. Reduced surface tension relative to the control PBS would indicate the presence of the wetting agents in the lens. Extended presence of the wetting agents would provide longer lasting wetting, better cleaning action and, consequently, reduced end-of-the-day dryness and improved overall comfort for lens wearers. For the three lens types tested, solution A outperformed solution B in providing a higher concentration and a longer release profile of surface-active agents. The study of continuous release of wetting agents from various lens materials is described in still greater detail in the example that follows.

### EXAMPLE 3

A. Lens Materials

Three materials were used in the subject continuous release of wetting agents from various lens materials study as described below.

Group I: Optima™ FW (−3.25 D) (Bausch & Lomb)

Group III: PureVision™ (−5.75 D) (Bausch & Lomb)

Group IV: Surevue™ (−7.00 D) (Johnson & Johnson)

B. Solutions

The multipurpose solutions used in the subject continuous release of wetting agents from various lens materials study are set forth below in Table 4.

TABLE 4

| Solution | Components | Weight Percent |
|---|---|---|
| A | Boric acid | 0.85 |
| | Sodium Phosphate (Monobasic) | 0.15 |
| | Sodium Phosphate (Dibasic) | 0.31 |
| | Sodium Chloride | 0.26 |
| | HAP (30%) | 0.10 |
| | Tetronic 1107 | 1.00 |
| | Pluronic F127 | 2.00 |
| | Polymer JR | 0.02 |
| | PHMB (20%) | 1.1 ppm |
| B including | Tetronic 1107 | 1.00 |

C. Procedure

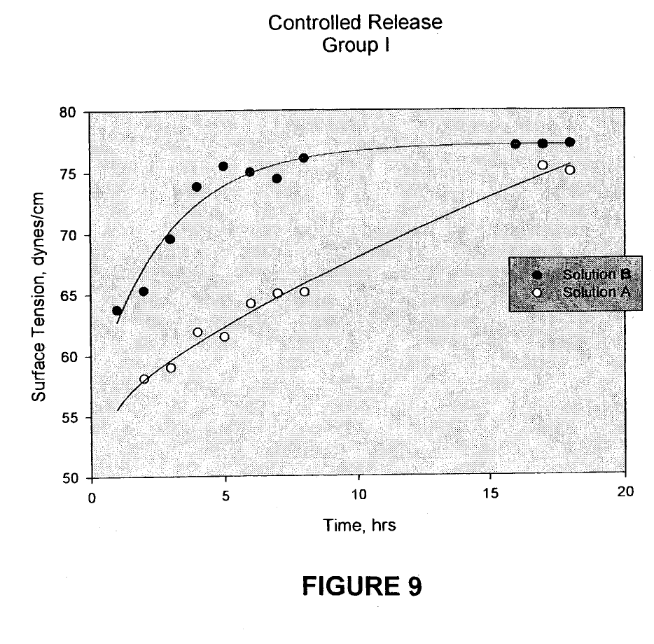

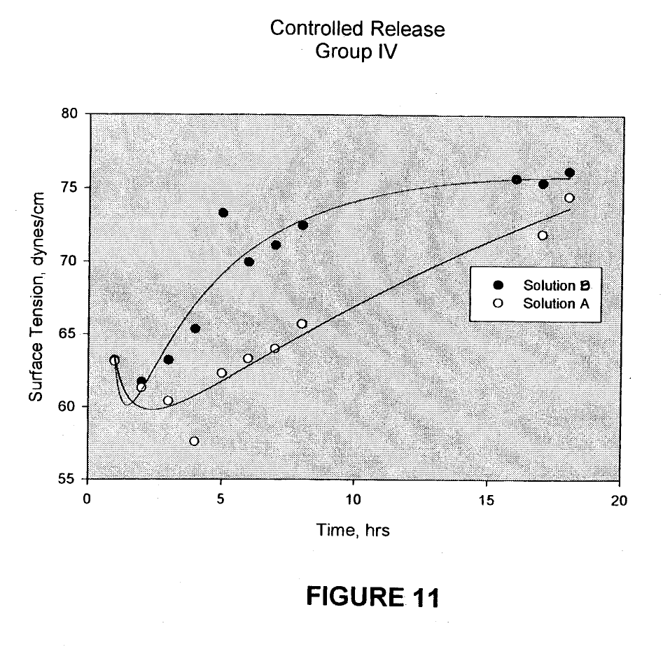

Various group type lenses were soaked for four hours in the test solutions A and B. The lenses were then removed and placed in a lens basket designed to receive a continuous infusion of phosphate buffered saline (PBS). A micro-infusion pump delivered 3.8 ml/min of PBS continuously to the lens surface for 18 hours to simulate the human tear film secretory rate in the eye. The solution dripping off the lens was collected over every hour for the first eight hours and then for the 16^{th}, 17^{th} and 18^{th} hour in a closed container to prevent evaporation. This volume was diluted with PBS to obtain 30 ml of solution. The apparent surface tension of the resulting solution was measured using the DuNouy ring method and the results were plotted as shown in FIGS. 9 through 11.

D. Results and Conclusions

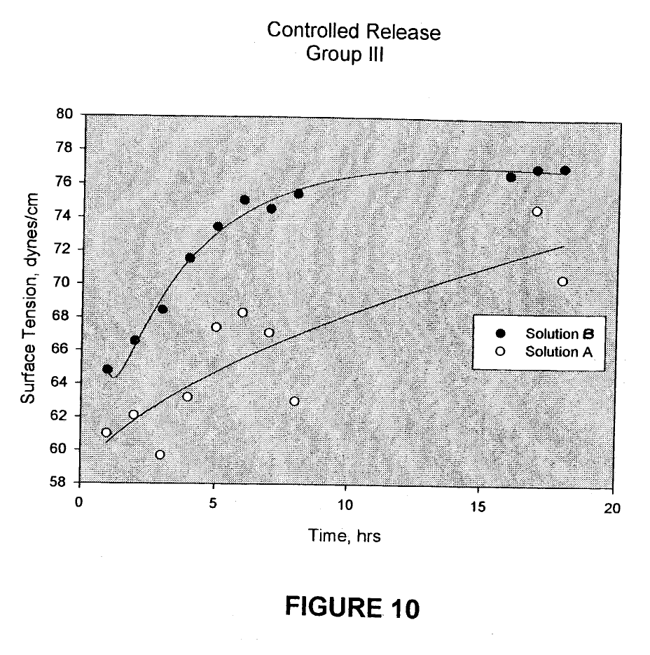

Non-linear regression models were used to fit the curves to the data collected. Since the surface tension is directly proportional to the concentration of surface active agents and since the elute volume was not exactly the same for each sample collected, some scatter in the surface tension data was expected. However, the trends illustrated in the graphs of FIGS. 9 through 11 are unmistakable. As illustrated in FIG. 9, test solution A showed a better release profile with the steeper slope over the first 8 hours presumably due to increased absorption characteristics of the wetting agents into the lens matrix than test solution B. As illustrated in FIG. 10, test solution A exhibited significantly better release profiles compared to test solution B tested over the first 8 hours implying that a greater amount of surface active agents was released in the eluted volume.

The wetting agents in test solution A most likely possess a stronger ability to penetrate the lens matrix and, due to the increased absorption, are more likely to demonstrate extended and controlled release of wetting agents in the eye. Such controlled release of wetting agents provides enhanced comfort for the lens wearer due to improved cleaning and longer lasting wetting.

Contact lenses from Group I, Group III and Group IV lens types were soaked in various solutions and frictional property measured using a highly sensitive Nano Scratch Tester at Micro Photonics, Inc., Irvine, Calif. Based on the study results, test solution A produced the lowest coefficient of friction (C of F) for all lens types than any other solution tested. Reduced coeffiecient of friction reduces lid friction over a contact lens in the eye during blinking and may contribute to improved overall comfort to the lens wearer. The polymers used as wetting agents in the test solution A formulation are most likely able to penetrate the lens matrix as well as “stack” on the lens surface to produce a smoother cushioned surface. The study of the coefficient of friction for various lens materials in multipurpose solutions is described in still greater detail in the example that follows.

### EXAMPLE 4

A. Lens Materials

Three materials were used in the subject coefficient of friction study as described below.

Group I: Optima™ FW (−3.25 D), Lot#R21000297, Exp. February 2005 (Bausch & Lomb)

Group III: PureVision™ (−3.75 D), Lot#R08000336 (Bausch & Lomb)

Group IV: SureVue™ (−7.00 D), Lot#291901, Exp. November 2006 (Johnson & Johnson)

B. Solutions

The multipurpose solutions used in the subject coefficient of friction study are set forth below in Table 5.

TABLE 5

| Solution | Components | Weight Percent |
|---|---|---|
| A | Boric acid | 0.85 |
| | Sodium Phosphate (Monobasic) | 0.15 |
| | Sodium Phosphate (Dibasic) | 0.31 |
| | Sodium Chloride | 0.26 |
| | HAP (30%) | 0.10 |
| | Tetronic 1107 | 1.00 |
| | Pluronic F127 | 2.00 |
| | Polymer JR | 0.02 |
| | PHMB (20%) | 1.1 ppm |
| B including | Tetronic 1107 | 1.00 |
| Control | Phosphate Buffered Saline | |

C. Procedure

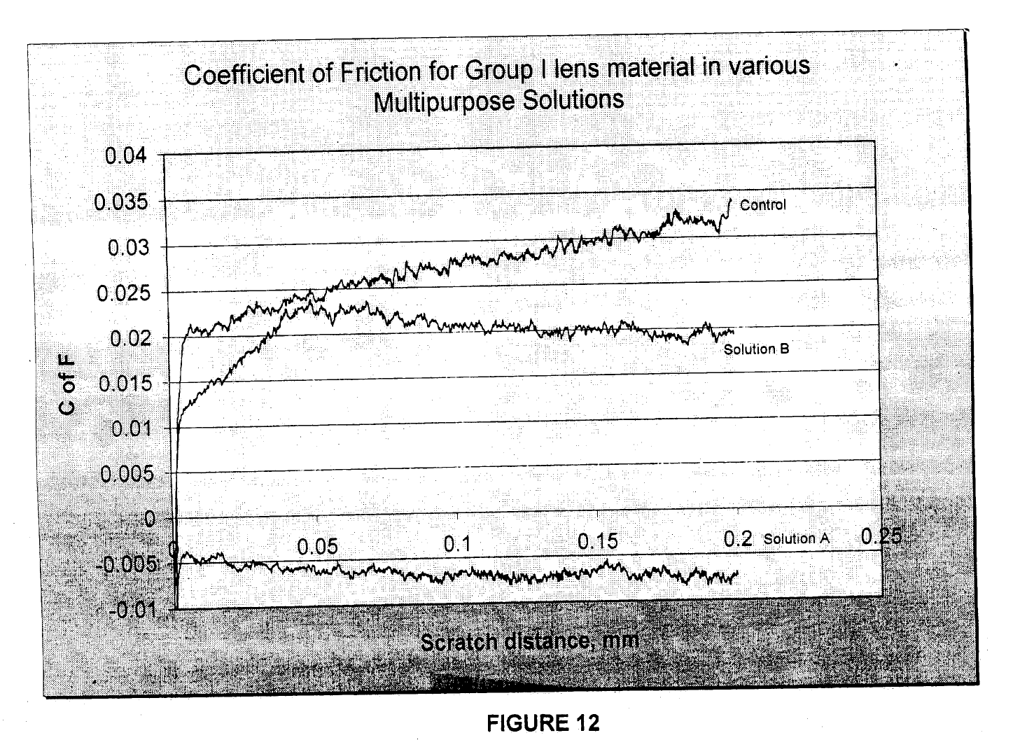

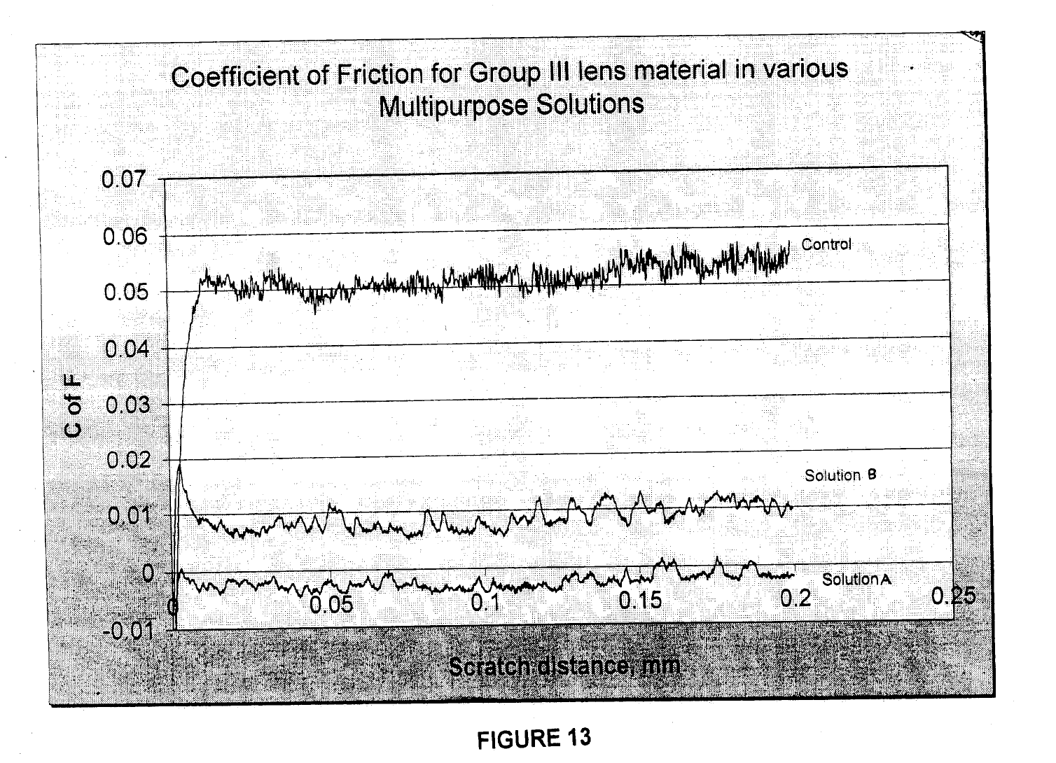

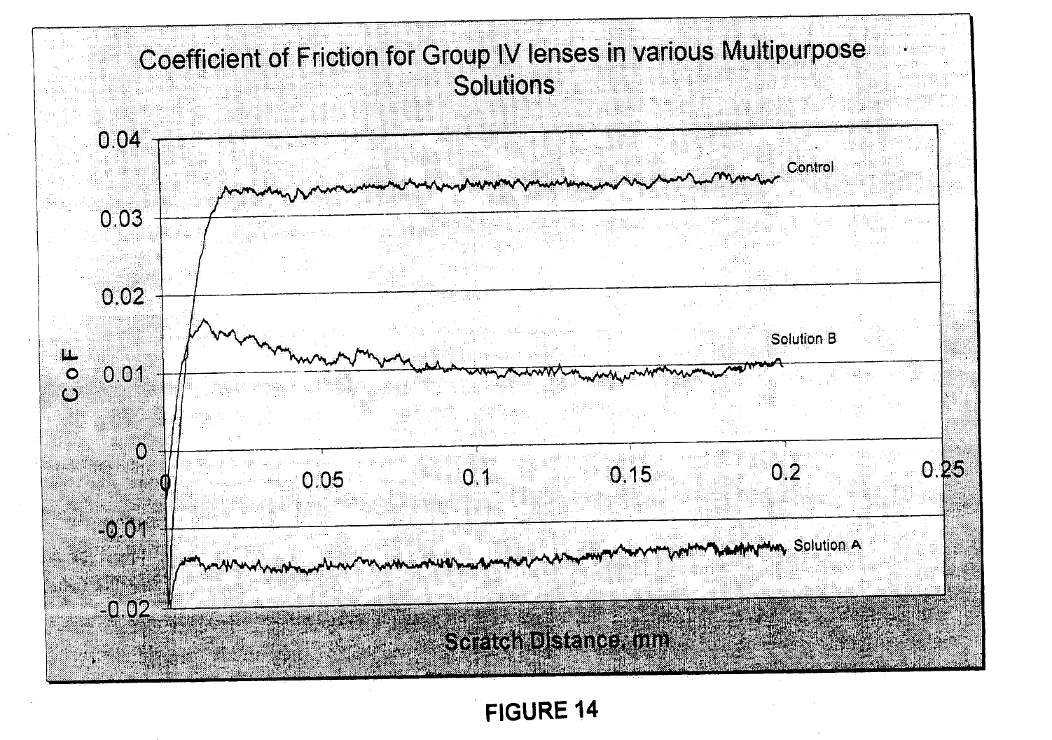

Contact lenses from Group I, Group III and Group IV lens types were soaked in each of the described solutions and frictional property measured as described above. The results obtained were plotted as shown in FIGS. 12 through 14.

D. Results

The results for test solution A for all lens types are at or below zero and is partly due to insufficient resolution of the friction table for the Nano Scratch Tester at friction values close to zero. Solution A exhibited the lowest coefficient of friction relative to the other solutions tested.

### EXAMPLE 5

A. Lens Materials

Lenses as described below were used in the subject polyether absorption study as described below.

Group IV: SureVue™, (Johnson & Johnson)

B. Solutions

Several test solutions were prepared by adding different concentrations of polyethers to the control solution described below in Table 6.

TABLE 6

| Solution | Components | Weight Percent |
|---|---|---|
| Control Solution | Boric acid | 0.85 |
| | Sodium Phosphate (Monobasic) | 0.15 |
| | Sodium Phosphate (Dibasic) | 0.31 |
| | HAP (30%) | 0.1 |
| | Sodium chloride | 0.26 |
| | PHMB | 1.1 ppm |

C. Procedure

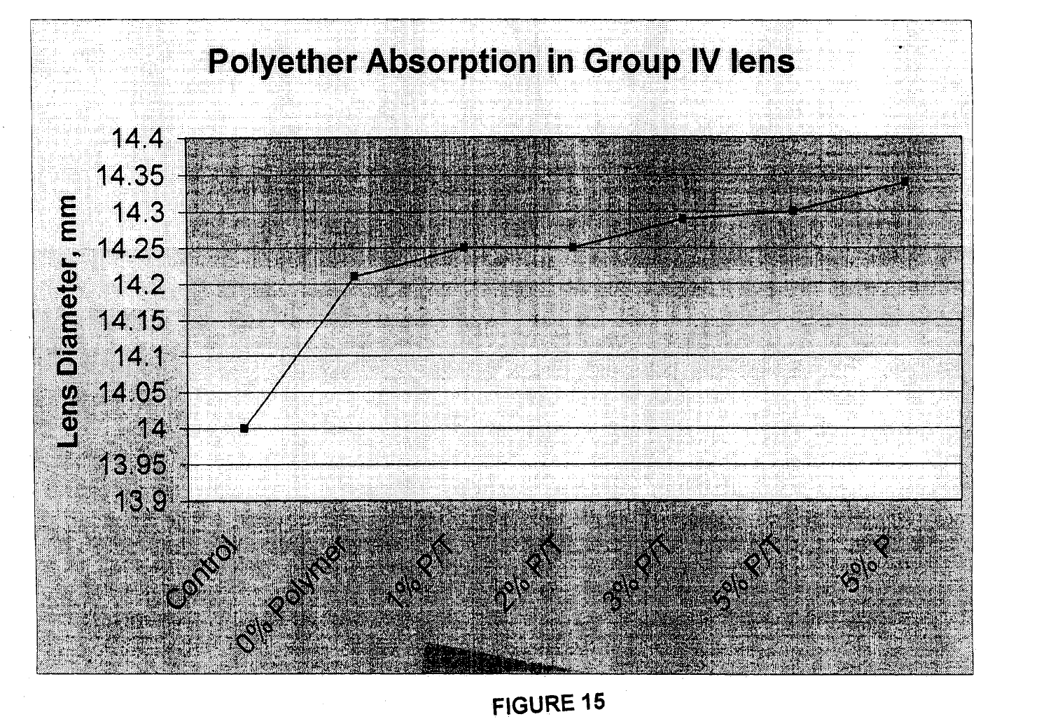

SureVue lenses were soaked in test solutions, prepared by adding different concentrations of polyethers to the above identified control solution, for four hours and then placed under a microscope. While under the microscope, the lenses were submerged in the same solution they were soaked in the previous four hours. Furthermore, the soaking was staggered in 5-minute intervals to assure that there was an equal amount of soaking. Using imaging software connected to the microscope, the lens diameter was measured. The microscope was first calibrated with a disc of known diameter (9.6 mm). SureVue lenses are 14.0 mm. The lens diameter data measured for each test solution is set forth below in Table 7 and illustrated in FIG. 15.

TABLE 7

| Solution | Lens diameter after 4 hour soak |
|---|---|
| Control solution (0% polyether) | 14.21 mm |
| Control solution + 1% P/T | 14.25 mm |
| Control solution + 2% P/T | 14.25 mm |
| Control solution + 3% P/T | 14.29 mm |
| Control solution + 5% P/T | 14.30 mm |
| Control solution + 5% P | 14.34 mm |

While there is shown and described herein ophthalmic solutions, hydrogel substrates and methods of making and using the same, it will be manifest to those skilled in the art that various modifications may be made without departing from the spirit and scope of the underlying inventive concept. The present invention is likewise not intended to be limited to particular ophthalmic solutions, substrates or methods described herein except insofar as indicated by the scope of the appended claims.

We claim:

1. An ophthalmic solution for absorption into and controlled release over time from hydrogel biomaterials comprising:
greater than about one weight percent polyethers in a buffered aqueous solution.

2. The ophthalmic solution of claim 1 wherein said solution contains about 1.5 to 14 weight percent polyethers.

3. The ophthalmic solution of claim 1 wherein said solution contains about 2 to 5 weight percent polyethers.

4. The ophthalmic solution of claim 1 wherein said solution has a pH of about 6.0 to 8.0.

5. The ophthalmic solution of claim 1 wherein said solution has a pH of about 6.5 to 7.8.

6. The ophthalmic solution of claim 1 wherein said solution includes about 0.1 to 1.5 percent by weight buffer.

7. The ophthalmic solution of claim 1 wherein said solution includes about 0.05 to 2.5 percent by weight buffer.

8. The ophthalmic solution of claim 1 wherein said solution includes one or more buffers selected from the group consisting of boric acid, sodium borate, potassium citrate, citric acid and sodium bicarbonate.

9. The ophthalmic solution of claim 1 wherein said solution includes one or more tonicity adjusting agents selected from the group consisting of sodium chloride, potassium chloride, dextrose, gycerin, calcium and magnesium chloride.

10. The ophthalmic solution of claim 1 wherein said solution includes about 0.01 to 2.5 percent by weight tonicity adjusting agent.

11. The ophthalmic solution of claim 1 wherein said solution includes one or more viscosity builders.

12. The ophthalmic solution of claim 1 wherein said solution includes poly(vinyl alcohol) as a viscosity builder.

13. The ophthalmic solution of claim 1 wherein said solution has an osmolality of about 200 to 400 mOsm/kg.

14. The ophthalmic solution of claim 1 wherein said polyethers are poly(ethylene oxide)-poly(propylene oxide)-poly(ethylene oxide) and poly(propylene oxide)-poly(ethylene oxide)-poly(propylene oxide).

15. A method of using the ophthalmic solution of claim 1 comprising: exposing a hydrogel biomaterial contact lens to said ophthalmic solution.

16. A method of making an ophthalmic solution for absorption into and controlled release over time from hydrogel biomaterials comprising: adding greater than about one weight percent polyethers to a buffered aqueous solution.

17. The method of claim 16 wherein about 1.5 to 14 weight percent polyethers are added.

18. The method of claim 16 wherein about 2 to 5 weight percent polyethers are added.

19. The method of claim 16 wherein said polyethers are poly(ethylene oxide)-poly(propylene oxide)-poly(ethylene oxide) and poly(propylene oxide)-poly(ethylene oxide)-poly(propylene oxide).

20. The method of claim 16 wherein one or more tonicity adjusting agents, one or more and optionally, one or more viscosity builders are added.

21. An ophthalmic solution for absorption into and controlled release over time from hydrogel biomaterials comprising: boric acid, monobasic sodium phosphate, dibasic sodium phosphate, sodium chloride, one or more polyethers at greater than one percent by weight, Polymer JR and a disinfectant agent.

22. An ophthalmic solution for absorption into and controlled release over time from hydrogel biomaterials comprising: a buffering system, one or more tonicity adjusting agents and one or more polyethers at greater than one percent by weight.
